(19) United States
(12) Patent Application Publication
(10) Pub. No.: US 2004/0204697 A1
(43) Pub. Date: Oct. 14, 2004

(54) Absorbent articles containing absorbent cores having at least one outer layer containing microwells

(76) Inventor: John D. Litvay, Downingtown, PA (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/411,376

(22) Filed: Apr. 11, 2003

(51) Int. Cl. A61F 13/15; A61F 13/20

(52) U.S. Cl. 604/367; 604/387

(57) ABSTRACT

The present invention relates generally to an absorbent core for an absorbent article, and more particularly to an absorbent core having at least one outer layer containing a plurality of microwells and optionally adhesives. Such absorbent cores provide the flexibility of creating precise zoning of particular properties throughout the core, and they provide improved comfort and fit.

### FIELD OF THE INVENTION

The present invention relates generally to an absorbent core for an absorbent article, and more particularly to an absorbent core having outer layers having microwells due to embossing. The microwells provide for additional retention of superabsorbent particles, as well as the potential for zoned absorbency due to specific placement of superabsorbent particles in the microwells. Such absorbent cores provide increased absorbency, additional flexibility of creating precise zoning of particular properties throughout the core, and they provide improved comfort and fit.

### BACKGROUND OF THE INVENTION

Disposable absorbent garments such as infant diapers or training pants, adult incontinence products and other such products typically were constructed with a moisture-impervious outer backing sheet, a moisture-pervious body-contacting inner liner sheet, and a moisture-absorbent core sandwiched between the liner and backing sheets. Much effort has been expended to find cost-effective materials for absorbent cores that display favorable liquid absorbency and retention. Superabsorbent materials in the form of granules, beads, fibers, bits of film, globules, etc., have been favored for such purposes. Such superabsorbent materials generally are polymeric gelling materials that are capable of absorbing and retaining even under moderate pressure large quantities of liquid, such as water and body wastes, relative to their own weight.

The superabsorbent material generally is a water-insoluble but water-swellable polymeric substance capable of absorbing water in an amount which is at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

The ability of a superabsorbent material to absorb liquid typically is dependent upon the form, position, and/or manner in which particles of the superabsorbent are incorporated into the absorbent core. Whenever a particle of the superabsorbent material and absorbent core is wetted, it swells and forms a gel. Gel formation can block liquid transmission into the interior of the absorbent core, a phenomenon called “gel blocking.” Gel blocking prevents liquid from rapidly diffusing or wicking past the “blocking” particles of superabsorbent (e.g., those particles that have swelled and touched an adjacent swelled particle), causing portions of a partially hydrated core to become inaccessible to multiple doses of urine. Further absorption of liquid by the absorbent core must then take place via a diffusion process. This is typically much slower than the rate at which liquid is applied to the core. Gel blocking often leads to leakage from the absorbent article well before all of the absorbent material in the core is fully saturated.

Despite the incidence of gel blocking, superabsorbent materials are commonly incorporated into absorbent cores because they absorb and retain large quantities of liquid, even under load. However, in order for superabsorbent materials to function, the liquid being absorbed in the absorbent structure must be transported to unsaturated superabsorbent material. In other words, the superabsorbent material must be placed in a position to be contacted by liquid. Furthermore, as the superabsorbent material absorbs the liquid it must be allowed to swell. If the superabsorbent material is prevented from swelling, it will cease absorbing liquids.

Adequate absorbency of liquid by the absorbent core at the point of initial liquid contact and rapid distribution of liquid away from this point is necessary to ensure that the absorbent core has sufficient capacity to absorb subsequently deposited liquids. Previously known absorbent cores have thus attempted to absorb quickly and distribute large quantities of liquids throughout the absorbent core while minimizing gel blocking during absorption of multiple doses of liquid.

In general, some of the important performance attributes of an absorbent core of a diaper (or any other absorbent garment) are functional capacity, rate of absorption, core stability in use, type of SAP, ratio of fibrous material to SAP, the type and basis weight of glue or tackifying agent used to adhere the SAP to the fibrous material or tissue wrapping, and the basis weight of the core. Absorption under load or AUL is a good measure of functional capacity and the rate at which that absorption occurs. AUL is believed to be a function of both SAP basis weight (mass per unit area) and the composition of SAP used in the composite. Increasing the basis weight decreases the performance/cost ratio of the absorbent core, making them uneconomical. Also, increased basis weights tend to affect the fit and comfort of the garment, as well as impacting the packaging and shipping costs.

It is known to provide absorbent laminates comprised of, for example, an upper and lower layers, and a central fibrous layer containing from 50% to 95% by weight SAP. U.S. Pat. No. 6,068,620, the disclosure of which is incorporated herein by reference in its entirety, discloses that the upper and lower layers are comprised of tissue, airlaid fluff pulp or synthetic non-woven fibrous layers. The upper and lower layers are said to assist in maintaining the integrity of the core, the laminate layered arrangement is said to minimize gel blocking, and the laminate can be folded in various configurations.

It also is known to provide absorbent cores comprised of differing materials in an attempt to maximize comfort and efficiency of the core, and to provide areas having varying degrees of absorbency. U.S. Pat. No. 5,849,002, the disclosure of which is incorporated by reference herein in its entirety, discloses absorbent cores having three zones: (i) one zone for receiving fluids; (ii) one zone for distributing and storing fluids; and (iii) one zone for preventing leakage. U.S. Pat. No. 5,853,402, the disclosure of which is incorporated by reference herein in its entirety, discloses composite absorbent cores comprising at least an absorbent material and a porous resilient material. Other composite, zoned, or multi-component cores are disclosed in, for example, U.S. Pat. No. 5,681,300 (blended absorbent core), U.S. Pat. No. 5,882,464 (crimping to join two absorbent structures), U.S. Pat. No. 5,891,120 (varying SAP concentration throughout core), U.S. Pat. Nos. 5,425,725 and 5,983,650 (multiple fiber free SAP pockets in core), and U.S. Pat. No. 5,922,165 (method of joining outer layers with absorbent core disposed between the outer layers). The respective disclosures of each of these documents are incorporated by reference herein in their entirety.

The disclosure herein of disadvantages and poor performance of known products, methods, and apparatus is not intended to limit the scope of the present invention. Indeed, various embodiments of the invention may include some of the known products, methods, and apparatus without suffering from the disadvantages.

### SUMMARY OF THE INVENTION

It would be desirable to provide an absorbent garment having an improved ability to retain fluids and consequently, to prevent leakage. It also would be desirable to provide an absorbent core that includes an increased amount of superabsorbent polymers, but at the same time does not suffer from gel blocking to an appreciable extent. A further desirable feature would be to provide an absorbent core having varying areas of absorbency to account for variations in gender and age, that is relatively easy and inexpensive to manufacture.

It therefore is a feature of an embodiment of the invention to provide an absorbent garment having an improved ability to retain fluids, especially in areas of the core where fluid retention is needed most. It is an additional feature of an embodiment of the invention to provide an absorbent garment that includes an absorbent core having SAP particles as a substantial percentage of its basis weight, but at the same time reducing gel blocking, i.e., retaining high SAP efficiency. An additional feature of the invention is to provide an absorbent article having specific desired properties in select areas of the absorbent core that is relatively inexpensive to manufacture, that provides the improved properties above, and that is comfortable to wear.

These and other features of the invention can be achieved by an absorbent article including a top sheet, a back sheet and an absorbent core disposed between the top sheet and the back sheet. The absorbent core of the invention preferably is comprised of an upper layer, a lower layer, and a central absorbent layer including a fibrous mixture of fibers and SAP disposed between the upper and lower layer. At least one of the upper and/or lower layer includes a plurality of microwells that contain at least a portion of the SAP. The microwells can be embossed with a pattern such that the absorbent core of the invention contains areas of varying absorbency due to the varying concentrations of SAP in the core.

In accordance with a feature of an embodiment of the invention, the absorbent core includes adhesives applied to at least the microwells. Such an absorbent core allows for lesser or greater amounts of absorbent material in select regions.

In accordance with an additional feature of an embodiment of the invention, there is provided a method of making an absorbent article that includes providing a top sheet material and a back sheet material. The method also include preparing an absorbent core that includes providing an optional upper layer and a lower layer and disposing between the upper and lower layers, a central absorbent layer including a mixture of fibers and SAP. At least one of the optional upper layer and lower layer is patterned prior to forming the absorbent core in such a manner that the layer(s) contains a plurality of microwells. The absorbent core then is disposed between the top sheet material and the back sheet material.

Preparing the absorbent core also may include supplying select regions of adhesive to at least one of optional upper and lower layers prior to disposing the central absorbent layer there between, to thereby provide an absorbent core having select regions of absorbency due to the presence of varying concentrations of SAP.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a partially cut-away view of an embodiment of the present invention, shown with elastic members fully stretched in the main portion of the garment;

FIG. 2 is an illustration of an apparatus useful in carrying out a method of making an absorbent garment in accordance with the present invention;

FIG. 3 is a top view of a lower layer including a pattern of a plurality of microwells;

FIG. 4 is a top view of a lower layer including another pattern of a plurality of microwells;

FIG. 5 is a cross-sectional view of an absorbent core in accordance with one embodiment of the invention, drawn along line A-A of FIG. 1;

### DETAILED DESCRIPTION OF THE PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein. Preferably, the absorbent core is thin in order to improve the comfort and appearance of a garment.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

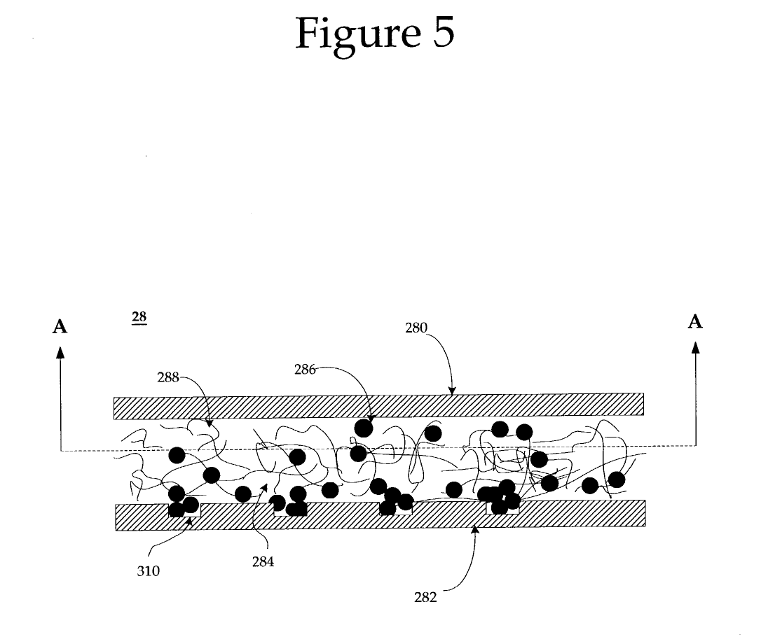

In addition, upper and lower layer refers to the ultimate configuration of the absorbent core, a preferred cross section of which is illustrated in FIG. 5. The upper and lower layers may be comprised of the same material folded over the central absorbent layer. Thus, in essence only one layer of material is used in forming the core.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the expression “tow fibers” relates in general to any continuous fiber. Tow fibers typically are used in the manufacture of staple fibers, and preferably are comprised of natural and/or synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow. Tow as used in the context of the present invention also encompasses modified tow fibers that have been either surface or internally modified (chemically or otherwise) to improve various desired properties of the fibers (e.g., wicking, etc.).

The present invention relates generally to absorbent articles, and in particular to an absorbent article that contains a top sheet, a back sheet, and an absorbent core disposed at least partially between the top sheet and the back sheet. The absorbent core of the invention preferably has varying concentrations of SAP throughout its cross-section, whereby the central absorbent layer is comprised of a fibrous mixture of fibers and SAP. The absorbent core preferably is comprised of outer layers and the central absorbent layer, whereby at least one of the outer layers includes microwells. The SAP is capable of migrating to and settling into the microwells, thereby enabling varying the concentration of SAP throughout the core by the particular arrangement of microwells. The microwells preferably are formed by embossing at least one of the outer layers.

The invention also relates in general to a method of making an absorbent article that includes providing a top sheet material and a back sheet material. The method also includes preparing an absorbent core that contains an upper layer, a lower layer, and at least a central absorbent layer comprised of a mixture of fibrous material and SAP disposed between the upper and lower layers. At least one of the upper layer or the lower layer contains microwells.

Preparing the absorbent core includes forming microwells in at least one of the upper layers or the lower layers. The microwells may be formed in at least one of the layers separately, or they may be formed just prior to forming the absorbent core. The method of forming the core optionally includes supplying select regions of adhesive to at least one of the upper and lower layers, preferably in or on the microwells or microwells-containing region, prior to disposing the central absorbent layer there between, to thereby provide an absorbent core having select regions of absorbency due to the presence of varying concentrations of SAP. In one embodiment of the invention, no adhesive is applied to the upper or lower layer thereby providing a gravitationally zoned absorbent core, whereby the SAP migrates into the microwells.

The absorbent article of the invention preferably has a front waist region, a rear waist region and a crotch region positioned between the front and rear waist regions. The front waist region and rear waist region can be associated with one another to form a waist opening, and two leg openings. Those skilled in the art recognize that “front” and “rear” in the context of the invention denote for clarity purposes only the front and rear of a user, and that the absorbent article could be reversed whereby the previously described “front” portion becomes the rear portion, and vice versa.

Leg elastics preferably are provided along the leg openings for securely holding the leg openings against the thighs of the wearer to improve containment and fit. A fastening system, either resealable or permanent, preferably holds the absorbent article around the wearer's waist. The fastening system assists in associating the front waist region with the rear waist region. A pair of stand-up leg gathers or waist containment flaps may be attached to or formed from the body's side surface of the top sheet.

The preferred embodiments of the absorbent article of the invention include an absorbent core comprising a mixture of tow fibers and SAP. The absorbent core has select regions of absorbency due to the presence or absence of mcirowells in the upper and/or lower layer. The absorbent core and/or the absorbent article also may include one or more additional components, such as at least one layer selected from an acquisition layer, a distribution layer, an additional fibrous layer containing SAP, a wicking layer, a storage layer, or combinations and fragments of these layers.

Other non-SAP-containing roll good materials such as latex or thermally bonded airlaid fluff pulp, (e.g., roll good available from Walkisoft, Merfin or Fort James), or synthetic spunbonded, carded, or hydro-entangled non-woven may be positioned above and below the absorbent core. The absorbent core also may be comprised of more than one absorbent core unit. The absorbent core of the invention preferably contains 50-95% by weight particulate or fibrous SAP and a tow fiber, which preferably is capable of maintaining high SAP efficiency. As described in U.S. Pat. No. 6,068,620, SAP efficiency can be expressed as the ratio of the actual SAP absorbency under load, or AUL (expressed as grams of saline absorbed per gram of SAP in the laminate), and the maximum SAP AUL obtained under ideal conditions of low basis weight where gel blocking does not occur. SAP concentrations of 50-95% provide thinner roll good composites for efficient shaping and handling. High SAP concentrations also provide thinner absorbent cores that can provide new options for product design. The absorbent core useful in the invention can be made using either a wet or dry process, but a dry process is particularly preferred.

The outer layers of the absorbent cores of the invention typically are designed for optimal wet/dry strength, liquid acquisition and distribution, as well as SAP containment. The inner layers of absorbent cores generally are designed for optimal absorbency and SAP efficiency. Designers of absorbent cores in the past have had to combine the attributes of the outer and inner layers into a homogeneous composite, often leading to an unacceptable compromise.

Absorbent cores made of fibrous materials, e.g., tow fibers and SAP typically include a tackifying agent or other type of material to adhere the SAP to the fibers, or to contain the SAP. Use of tackifying agents and/or adhesives to adhere the SAP to the fibers or to the microwells, however, can have an adverse effect on the absorbency properties of the SAP, and can cause excessive gel blocking. Traditional cores also make it difficult to vary the absorbency throughout the cross-section of the absorbent core. These conventional cores typically were designed with a single basis weight, a single type of SAP, a single ratio of fiber to SAP, a single glue basis weight, and a single glue type. Varying any of these parameters throughout the length and/or width of the absorbent core is not practical from a manufacturing standpoint.

The present invention is premised in part on the discovery that varying amounts of SAP can be provided throughout the cross-section of the absorbent core by providing microwells only in select regions on the upper and/or lower layers of the absorbent core, and by optionally applying adhesives at or near the microwells or microwells-containing regions. No adhesives may be applied in one preferred embodiment. By providing microwells in select regions of the upper and/or lower layers, more SAP will migrate to the upper and/or lower layers in the areas where the microwells are present, (see, e.g., FIG. 5), thereby providing enhanced absorbency in those areas. In addition, providing the microwells to the upper and/or lower layers is easier and less problematic from a manufacturing perspective than spraying adhesives onto the fibers and SAP during manufacture of the central layer, or than varying the type and/or amount of SAP or fiber supplied to the core. Applying the microwells and optional adhesive in this manner also is not believed to significantly coat the SAP particles entirely, which in turn does not adversely affect the absorbency properties of the SAP as much as it would were the adhesive applied to the fibers and/or SAP during formation of the central absorbent layer. The optional adhesives can be provided to the microwells or microwells-containing regions in such a manner by select positioning of adhesive spray nozzles, by controlling the timing of the spray nozzle activation, and by other methods recognized by those having ordinary skill in the art.

The invention now will be described with reference to the attached drawings illustrating preferred embodiments of the invention. For clarity, features that appear in more than one Figure have the same reference number in each Figure.

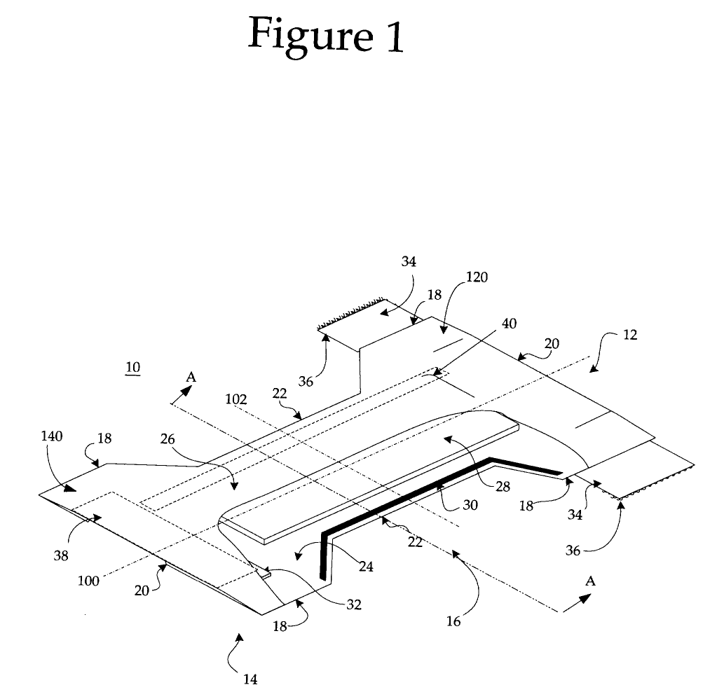

FIG. 1 is a partially cut away depiction of an exemplary embodiment of an absorbent garment 10 (preferably a disposable absorbent garment) of the present invention. The embodiment shown in FIG. 1 is an infant's diaper, however, this depiction is not intended to limit the invention, and those skilled in the art appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their relaxed condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 form the perimeter of a pair of leg openings, when the garment 10 is placed on a subject wearer.

The garment 10 preferably comprises a top sheet 24, and a back sheet 26, which may be substantially coterminous with the top sheet 24. When the garment 10 is being worn, the top sheet 24 faces the wearer's body, and the back sheet 26 faces away from the wearer. An absorbent core 28 preferably is disposed between at least a portion of the top sheet 24 the back sheet 26.

An embodiment of the present invention may further comprise various additional features. One or more pairs of elastic gathers 30 may extend adjacent the crotch edges 22. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of elastic waist foam 32 or other elastically extensible material, which help contract the garment around the wearer's waist, providing improved fit and leakage prevention.

The absorbent garment 10 also preferably includes fastening elements to enable attachment of the first waist region 12 to second waist region 14. Fastening elements preferably include a pair of tabs 34 that extend laterally away from opposite lateral edges 18 of the first waist region 12 of the garment 10. The tabs 34 may comprise an elastically extensible material (not shown), and may be designed to stretch around a wearer's waist to provide improved fit, comfort, and leakage protection. Such elasticized tabs 34 may be used in conjunction with, or in lieu of, waist foam 32, or other elastically extensible materials 32.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14.

Although not shown in the drawings, the absorbent garment 10 may also include grips attached along one of its edges proximal to each tab 34 to enable a caregiver to pull the grips, and not on the ends of the tabs 34, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The top sheet 24 and back sheet 26 may be constructed from a wide variety of materials known in the art. The invention is not intended to be limited to any specific materials for these components. The top sheet 24 and back sheet can be shaped and sized according to the requirements of each of the various types of absorbent garment, or to accommodate various user sizes. In an embodiment of the invention in which the garment 10 is a diaper or an adult incontinence brief, the combination of top sheet 24 and back sheet 26, may have an hourglass shape, as seen in FIG. 1, or may have a rectangular, trapezoidal, “T” shape, or other shape.

Due to the wide variety of backing and liner sheet construction and materials currently available, the invention is not intended to be limited to any specific materials or constructions of these components. The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 26 may be pigmented with, for example, titanium dioxide, to provide the garment 10 with a pleasing color or to render the back sheet 26 opaque enough that exudates being contained by the garment 10 are not visible from outside the garment. In addition, the back sheet 26 may be formed in such a manner that it is opaque, for example, by using various inert components in the polymeric film and then biaxially stretching the film. Other back sheet materials will be readily apparent to those skilled in the art. The back sheet 26 preferably has sufficient liquid imperviousness to prevent any leakage of fluids. The required level of liquid imperviousness may vary between different locations on the garment 10.

The back sheet 26 may further comprise separate regions having different properties. In a preferred embodiment, portions of the back sheet 26 are air-permeable to improve the breathability, and therefore comfort, of the garment 10. The different regions may be formed by making the back sheet 26 a composite of different sheet materials, chemical treatment, heat treatment, or other processes or methods known in the art. Some regions of the back sheet 26 may be fluid pervious. In one embodiment of the invention, the back sheet 26 is fluid impervious in the crotch 16, but is fluid pervious in portions of the first and second waist regions 12, 14. The back sheet 26 may also be made from a laminate of overlaid sheets of material.

The moisture-pervious top sheet 24 can be comprised of any suitable relatively liquid-pervious material known in the art that permits passage of liquid there through. Non-woven liner sheet materials are exemplary because such materials readily allow the passage of liquids to the underlying absorbent core 28. Examples of suitable liner sheet materials include non-woven spun bond or carded webs of polypropylene, polyethylene, nylon, polyester and blends of these materials.

The back sheet 26 may be covered with a fibrous, non woven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded non woven web of synthetic fibers such as polypropylene, polyethylene or polyester fibers; a non woven web of cellulosic fibers, textile fibers such as rayon fibers, cotton and the like, or a blend of cellulosic and textile fibers; a spun-bonded non woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet 26 may comprise three panels wherein a central poly back sheet panel is positioned closest to absorbent core 28 while outboard non-woven breathable side back sheet panels are attached to the side edges of the central poly back sheet panel. Alternatively, the back sheet 26 may be formed from microporous poly coverstock for added breathability.

The top sheet 24 also may be formed of three separate portions or panels. Those skilled in the art will recognize, however, that top sheet 24 need not be made of three separate panels, and that it may be comprised of one unitary item. A first top sheet panel may comprise a central top sheet panel formed from preferably a liquid-pervious material that is either hydrophobic or hydrophilic. The central top sheet panel preferably extends from substantially the second waist region 14 to the first waist region 12, or a portion thereof. The second and third top sheet panels (e.g., outer top sheet panels), in this alternative embodiment may be positioned laterally outside of the central top sheet panel. The outer top sheet panels preferably are substantially liquid-impervious and hydrophobic, preferably at least in the crotch area. The outer edges of the outer top sheet panels may substantially follow the corresponding outer perimeter of the back sheet 26. The material for the outer top sheet portions or panels is preferably polypropylene and can be woven, non-woven, spunbonded, carded or the like, depending on the application.

The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, either adhesively or thermally bonded, perforated plastic film, spun bonded fibers, or water entangled fibers, which generally weigh from 0.3-0.7 oz./sq. yd. and have appropriate and effective machine direction and cross-machine direction strength suitable for use as a baby diaper cover stock material.

The standing leg gather(s) preferably are disposed such that they extend laterally away from the surface of top sheet 24. Standing leg gather(s) 40 may be treated with a suitable surfactant to modify their hydrophobicity/hydrophilicity as desired, and they may be treated with skin wellness ingredients to reduce skin irritation. Alternatively, the standing leg gather(s) 40 may be formed as separate elements and then attached to the body side liner. The standing leg gather(s) 40 preferably include a portion that folds over onto itself to form a small enclosure. At least one, and depending on the size of the enclosure sometimes more than one, elastic member may be secured in the enclosure in a stretched condition. It is known that when the flap elastic attempts to assume the relaxed, unstretched condition, the standing leg gather(s) 40 rise above the surface of the top sheet 24.

The top sheet 24 may be made of any suitable relatively liquid-pervious material currently known in the art or later discovered that permits passage of a liquid there through. Examples of suitable top sheet materials include non woven spun-bonded or carded webs of polypropylene, polyethylene, nylon, polyester and blends of these materials, perforated, apertured, or reticulated films, and the like. Non woven materials are exemplary because such materials readily allow the passage of liquids to the underlying absorbent core 28. The top sheet 24 preferably comprises a single-ply non woven material that may be made of carded fibers, either adhesively or thermally bonded, spun bonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 24, and other top sheet materials will be readily apparent to those skilled in the art.

The top sheet 24 may further comprise several regions having different properties. In one embodiment of the present invention, the laterally distal portions of the top sheet 24, especially those used to make second and third top sheet panels, preferably are substantially fluid impervious and hydrophobic, while the remainder of the top sheet 24 (e.g., central top sheet panel) is hydrophilic and fluid pervious. Different top sheet properties, such as fluid perviousness and hydrophobicity, may be imparted upon the top sheet 24 by treating the top sheet 24 with adhesives, surfactants, or other chemicals, using a composite of different materials, or by other means. The top sheet 24 may also be made from a laminate of overlaid sheets of material. The top sheet 24 also may be treated in specific areas like the crotch region, with skin wellness ingredients such as aloe, vitamin E, and the like.

As noted elsewhere herein, the top sheet 24 and back sheet 26 may be substantially coterminous, or they may have different shapes and sizes. The particular design of the top sheet 24 and back sheet 26 may be dictated by manufacturing considerations, cost considerations, and performance considerations. Preferably, the top sheet 24 is large enough to completely cover the absorbent core 28, and the back sheet 26 is large enough to prevent leakage from the garment 10. The design of top sheet 24 and back sheet 26 is known in the art, and a skilled artisan will be able to produce an appropriate top sheet 24 and an appropriate back sheet 26 without undue experimentation.

The top sheet 24 and the back sheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, New Jersey, is used to join the top sheet 24 to the back sheet 26. The particular joining method may be dictated by the types of materials selected for the top sheet 24 and back sheet 26.

As mentioned above, absorbent garment preferably is provided with leg elastics 30 extending through crotch region 16, adjacent crotch edge 22. The absorbent garment of the invention also preferably is provided with waist elastic material 32 optionally in the first and second waist regions, 12, 14, respectively, to enable and assist in stretching around the wearer. The waist elastics 32 may be similar structures or different to impart similar or different elastic characteristics to the first and second waist regions 12, 14 of the garment. In general, the waist elastics may preferably comprise foam strips positioned at the first and second waist regions 12, 14, respectively. Such foam strips preferably are about ½ to about 1½ inches wide and about 3-6 inches long. The foam strips preferably are positioned between the top sheet 24 and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips 32 preferably are stretched 50-150%, preferably 100% more than their unstretched dimension before being adhesively secured between the back sheet 26 and top sheet 24.

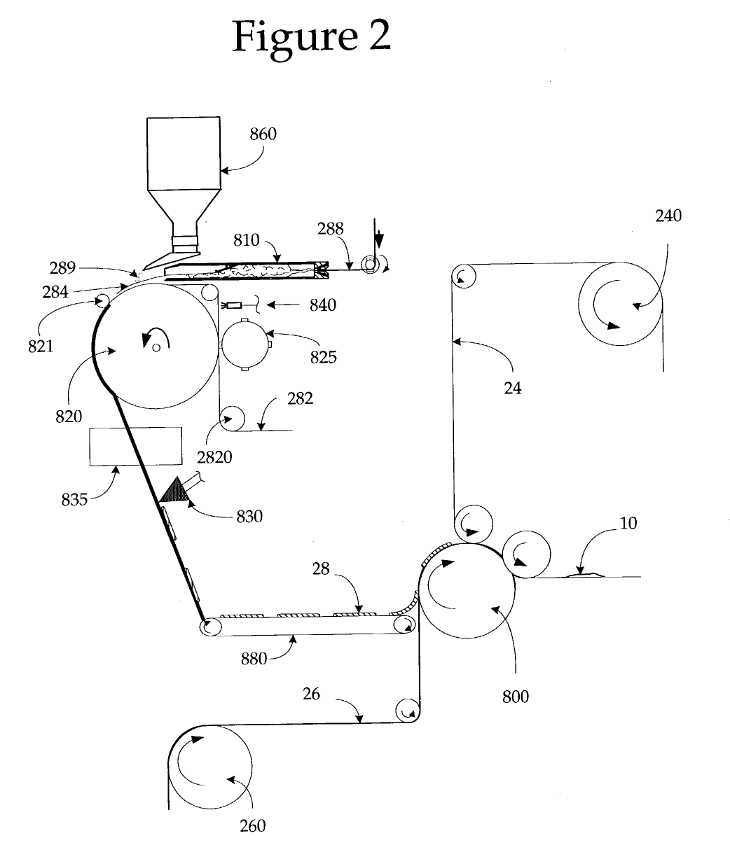

Each edge 22 that forms the leg openings preferably is provided with an adjacent leg elastic containment system 30. In the preferred embodiment, three strands of elastic threads (only two strands are shown in FIG. 2 for purposes of clarity) are positioned to extend adjacent to leg openings between the outer top sheet portions or panels and the back sheet 26. Any suitable elastomeric material exhibiting at least an elongation (defined herein as (Ls-LR)/LR where Ls is the stretch length of an elastic element and LR is retracted length, multiplied by 100 to obtain percent elongation) in the range of 5%-350%, preferably in the range of 200%-300%, can be employed for the leg elastics 30. The leg elastics 30 may be attached to the absorbent article 10 in any of several ways which are known in the art. For example, the leg elastics 30 may be ultrasonically bonded, heat/pressure sealed using a variety of bonding patterns, or glued to the garment 10. Various commercially available materials can be used for the leg elastics 30, such as natural rubber, butyl rubber or other synthetic rubber, urethane, elastomeric materials such as LYCRA (DuPont), GLOSPAN (Globe) or SYSTEM 7000 (Fulflex).

The fastening elements, preferably a fastening system 34 (e.g., tab 34) of the preferred embodiment, is attached to the first waist region 12, and it preferably comprises a tape tab or mechanical fasteners 36. However, any fastening mechanism known in the art will be acceptable. Moreover, the fastening system 34 may include a reinforcement patch below the front waist portion so that the diaper may be checked for soiling without compromising the ability to reuse the fastener. Alternatively, other absorbent article fastening systems are also possible, including safety pins, buttons, and snaps.

As stated previously, the invention has been described in connection with a diaper. The invention, however, is not intended to be limited to application only in diapers. Specifically, the absorbent cores of the preferred embodiments may be readily adapted for use in other absorbent garments besides diapers, including, but not limited to, training pants, feminine hygiene products and adult incontinence products.

The underlying structure beneath the top sheet 24 may include, depending on the diaper construction, various combinations of elements, but in each embodiment, it is contemplated that the absorbent garment will preferably include a absorbent core 28. In addition, an additional layer may be disposed between the top sheet 24 and absorbent core 28, and/or other additional layers may be disposed between these layers, or between absorbent core 28 and back sheet 26. An additional layer also may be included in the absorbent core 28. The additional layer(s) 29 may include a fluid transfer layer, a fluid handling layer, a storage layer, a wicking layer, a fluid distribution layer, and any other layer(s) known to those having ordinary skill in the art.

Although the absorbent core 28 depicted in FIG. 1 has a substantially rectangular cross-sectional and plan view shape, other shapes may be used, such as a “T” shape or an hourglass shape. The shape of the absorbent core 28 may be selected to provide the greatest absorbency with a reduced amount of material. The absorbent core may be associated with the top sheet 24, back sheet 26, or any other suitable part of the garment 10 by any method known in the art, in order to fix the absorbent core 28 in place. In addition to the respective layers in the absorbent core 28, the overall absorbent core 28 may be enclosed within a tissue wrapping, as disclosed in U.S. Pat. No. 6,068,620, the disclosure of which is incorporated by reference herein in its entirety. Skilled artisans are capable of designing and wrapping a suitable absorbent core 28 of the invention, using the guidelines provided herein.

The absorbent core 28 may extend into either or both of the first and second waist regions 12, 14. The absorbent core 28 of one preferred embodiment of the invention preferably includes at least three (3) layers whereby two of the layers are outer layers, 280, 282, FIG. 5, preferably outer tissue layers 280, 282, and an inner central fibrous layer 284, which preferably contains a mixture of fibrous material and SAP.

Upper and lower layers 280, 282 can be made of any suitable material capable of containing the inner layer(s) of absorbent core 28. Preferably, upper layer 280 is hydrophilic and fluid pervious, and lower layer 282 is hydrophobic and fluid impervious. It is preferred that upper and lower layers 280, 282 be comprised of a material selected from the group consisting of tissue, airlaid fluff pulp and synthetic non-woven materials. More preferably, upper layer 280 and lower layer 282 are comprised of the same tissue-like material. In various embodiments of the invention, upper layer 280 and lower layer 282 are made of different materials. In other embodiments of the invention, upper layer 280 and lower layer 282 are made of the same material and provided separately, or are made from the same single material and folded to include the central fibrous layer 284.

In a preferred embodiment, the central fibrous layer 284 of absorbent core 28 comprises super absorbent polymer distributed within a fibrous structure. Central fibrous layers 284 of this type generally are known in the art, and exemplary absorbent cores are described in U.S. Pat. No. 6,068,620 and U.S. Pat. No. 5,281,207, both issued to Chmielewski, and U.S. Pat. No. 5,863,288, issued to Baker, the disclosures of each of which are herein incorporated by reference in their entirety and in a manner consistent with this disclosure.

Certain fibrous and particulate additives preferably are used as constituent elements of the absorbent core 28 to maintain high SAP efficiencies when the SAP concentration is in the range of about 50-95%, more preferably about 60-90%, and most preferably about 75-85%. Fibrous additives of central fibrous layer 284 preferably include, but are not limited to, cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof. Of the foregoing, cellulose acetate is the most preferred fibrous additive for use in central fibrous layer 284. In addition, rayon, Courtauld's LYOCELL, polyacrylonitrile, cotton fibers and cotton linters have similar properties to cellulose acetate and are alternatively preferred. The remaining fibers, surface-modified polyolefin/polyester bicomponent fibers, and surface-modified polyester/polyester bicomponent fibers are also believed to be effective fibrous additives.

The fibrous component of the central layer 284 of absorbent core 28 preferably is comprised of tow fiber, and most preferably is a crimped tow of cellulose acetate, polypropylene, polyester, or mixtures thereof. Before making the absorbent core that includes a tow fiber, the tow fiber typically is unwound and opened, and then fed to the core forming station to provide a fibrous mass of material (see, FIG. 2). Skilled artisans are aware of techniques available to open tow fibers and form the opened fibers into a fibrous mass. In addition, the fibrous component of the central fibrous layer 284 may include a low-density roll good made in a separate process. Still further yet, the fibrous component could also include a carded web formed on-line. Optionally, it is advantageous to introduce from about 1-5% of a thermally bondable fiber into the fibrous component of the central fibrous layer 284 for wet strength and core stability in use. In addition to the tow material used as the fibrous component in central fibrous layer 284, other fibrous components also may be used.

To maintain high SAP concentrations, the concentration of fibrous material in the central layer 284 of the absorbent core 28 of the invention preferably is about 5-50%, more preferably about 10-30%, and most preferably about 15-25%. Most preferably, the central fibrous layer 284 comprises from about 75-85% SAP and from about 15-25% fibrous materials selected from the foregoing group, or the fibrous components discussed below.

Particulate additives may be added to central fibrous layer 284 in addition to or as a substitute for the foregoing fibrous additives in order to maintain high SAP efficiency. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity. hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent laminates. Examples of SAP/particulate and SAP/fiber/particulate additives include those described in, for example, U.S. Pat. No. 6,068,620.

Any superabsorbent polymer (SAP) now known or later discovered may be used in central fibrous layer 284 so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Examples of suitable SAP are water swellable polymers of water soluble acrylic or vinyl monomers crosslinked with a polyfunctional reactant.

Also included are starch modified polyacrylic acids and hydrolyzed polyacrylonitrile and their alkali metal salts. A more detailed recitation of superabsorbent polymers is found in U.S. Pat. No. 4,990,541 to Nielsen, the disclosure of which is incorporated herein by reference in its entirety.

Commercially available SAPs include a starch modified superabsorbent polymer available under the trade name HYSORB® from BASF Aktiengesellschaft, Ludwigshafen, Germany. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP, and AQUA KEEP SA60S, manufactured by Sumitomo Seika Chemicals Co., Ltd., Osaka Japan.; ARASORB manufactured by Arakawa Chemical (U.S.A.) Inc.; FAVOR manufactured by Stockhausen Inc.; DIAWET, commercially available from Mitsubishi Chemicals, Japan; FLOSORB, available from SNF Floerger, France, AQUALIC, available from Nippon Shokubai, Osaka, Japan.

In accordance with the present invention, the absorbent core preferably comprises a tow fiber, and preferably, a continuous crimped filament tow. This fiber structure has high structural integrity, and as such, is distinct from a matrix of discontinuous fibers described as fluff, or fluff pulp in the prior art. The high structural integrity enables the production of stronger webs than those formed from discontinuous fibers, which in turn are believed to enable the production of thinner absorbent pads. In addition, the use of such fibers enables the production of ultra low density absorbent cores, when compared to absorbent cores prepared by dispersing SAP particles in fluff.

The tow fiber can be any continuous or discontinuous thermoplastic filament tow fiber that is capable of being opened and used in combination with SAP in an absorbent core. Preferably, polypropylene and cellulose ester tow is used as the fibrous material in central fibrous layer 284. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include some ability to absorb moisture, (but absorptive capacity is not necessarily required), preferably is biodegradable, and is influenced not only by the substituent groups but also by the degree of substitution. The relationship between substituent groups, degree of substitution and biodegradability is discussed in W. G. Glasser et al, BIOTECHNOLOGY PROGRESS, vol. 10, pp. 214-219 (1994), the disclosure of which is incorporated herein by reference in its entirety.

Continuous filament tow useful in the present invention is beneficially moisture-absorbent and biodegradable. Accordingly, cellulose acetate tow is typically preferred for use in the invention. Typically, the denier per fiber (dpf) of the tow fiber will be in the range of about 1 to 25, preferably about 3 to 15, and most preferably about 4. For the same weight product, filaments of lower dpf may provide increased surface area and increased moisture absorption. Total denier may vary within the range of about 20,000 to 60,000, more preferably from about 25,000 to about 50,000, and most preferably from about 30,000 to about 40,000, depending upon the process used.

It is particularly preferred in the invention to use tow having crimped filaments. Tow materials having crimped filaments are typically easier to open. Separation of filaments resulting from bloom advantageously results in increased available filament surface area for superabsorbent material immobilization and increased moisture absorption. Gel blocking also may be reduced by using crimped tow in the central fibrous layer 284. As therefore may be understood, more crimp is typically better, with in excess of about 20 crimps per inch being usually preferred. Continuous filament, cellulose ester tow having crimped filaments with about 25 to 40 crimps per inch, is commercially available from Hoechst Celanese Corporation, Charlotte, N.C.

It is preferred in the present invention that the tow fibers in central fibrous layer 284 have an average length generally about the same length as the absorbent core. Typically, the two is a continuous filament that is cut to length during manufacture of the core. The average diameter of the tow fibers typically is expressed as the cross sectional area of the fibers, although the width of the fibers preferably is within the range of from about 50 to about 200 mm, more preferably from about 75 to about 150 mm, and most preferably from about 85 to about 120 mm. The cross sectional area is based on the denier and density of the fibers. For example, the denier per foot (dpf) and density (typically an acetate polymer density is about 1.32 g/cm³), can be used to calculate the cross sectional area. A 3.0 dpf acetate polymer fiber has a cross sectional area 2.525×10⁻⁶ cm².

It is preferred in the invention to use relatively coarse fibers having a low basis weight such that the pore size of the matrix formed by the mass of tow fibers does not entrain some or most of the SAP, but rather allows the SAP to fall freely through the matrix. The basis weight of preferred fibers used in the present invention ranges from about 20 to about 200 g/m², more preferably from about 50 to about 100 g/m², and most preferably from about 70 to about 80 g/m².

If desired, a superabsorbent, absorptive pad of multiple layer thickness, may be provided. To this end, the tow may be, for example, lapped or crosslapped in accordance with conventional procedures. In this way, a superabsorbent, absorptive material of a desired weight and/or thickness may be provided. The specific weight or thickness will depend upon factors including the particular end use. It is especially preferred that the crimped cellulose acetate tow material be opened and then mixed with the SAP particles to form the central fibrous layer 284.

The SAP may be provided in any particle size, and suitable particle sizes vary greatly depending on the ultimate properties desired. It has been known to prepare absorbent cores comprised of cellulose acetate tow or other polymeric fibers and SAP, as described in H1565, and U.S. Pat. Nos. 5,436,066, and 5,350,370, the disclosures of each of which are incorporated by reference herein in its entirety. It was conventional to add tackifying agents, specific size fibers, or specific fibers in combination with fluff, in order to prepare the absorbent core and immobilize the SAP particles. By adding these components during mixing of the fibrous material and SAP, it is difficult to control the concentration of either fiber or SAP across the cross-sectional area of the absorbent core 28. The present invention provides this flexibility by utilizing outer layers having microwells, and/or adhesives at or near the microwells, which in turn affects the distribution of fiber and SAP throughout the cross-sectional area of the absorbent core 28.

The total basis weight of the absorbent core 28 including fibrous materials, SAP, outer layers, additional layers, and additives, can be anywhere from about 50-1,000 grams per square meter. The most preferred total basis weights of the absorbent core 28 are about 400-700 grams per square meter. The absorbent core of an embodiment of the invention also may not include any SAP, but rather may include only tow fibers and perhaps other absorbent materials. These types of materials can be used as “swimmers,” or absorbent articles that can be used by children or adults in the water.

Optionally, about 1-10%, preferably about 5%, by weight of thermally bondable synthetic fibers can be added to the absorbent core 28 to impart additional wet strength to the laminate. This will improve the stability of the core during use of the diaper. The preferred synthetic fibers are polyolefin/polyester fibers and polyester/polyester bicomponent fibers.

The foregoing absorbent cores 28 of the preferred embodiments preferably are made using a dry process, whereby the respective components of the composite core 28 are brought together in a dry state, as opposed to one or more components being in a liquid state. Skilled artisans will be capable of making the absorbent cores 28 of the present invention, using the guidelines provided herein.

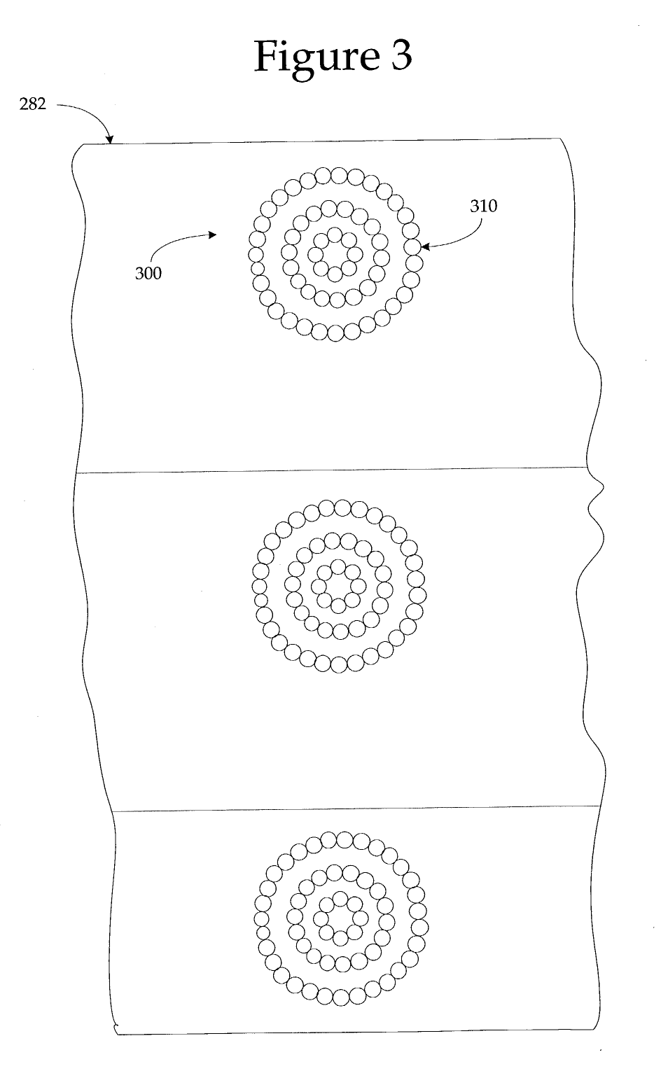

One of the features of the invention is that absorbent core 28 be made by providing microwells in either upper or lower layer 280, 282, or both, in varying sizes and at varying locations. The microwells can be designed such that the precise location and size applied to either upper or lower layer 280, 282, or both can be varied during a manufacturing run. In addition, the same embossing device can be used to form the same pattern of microwells, but adhesives can be applied at various locations during the manufacturing run to vary the concentration of the SAP. Accordingly, absorbent garments can be made having varying zones of absorbency without having to shut down the operation. For example, the size and location of the microwells 310 (FIGS. 3-5) and/or adhesives applied thereto or thereon can be located for optimum absorbency at or about the male insult point (e.g., about 10 cm from the bottom fold in a Stage 4 diaper) for a series of garments, and then changed for optimum absorbency at or about the female insult point (e.g., about 5 cm from the bottom fold in a Stage 4 diaper). Skilled artisans know where the male and female insult points are for the various stages of absorbent garment. Using the guidelines provided herein, a person skilled in the art therefore can design a suitable system to selectively provide microwells to upper or lower layer 280, 282, or both, and to provide absorbency zones for a variety of different types of garments (e.g., males, females, young, old, overnight, etc.).

The upper and lower layer 280, 282, encase the central absorbent layer 284, opened tow and SAP composite, and preferably form the upper and lower tissue layers of the completed garment, but may also form the top sheet and back sheet of the absorbent garment, or any other layers. The upper and lower layers 280, 282, preferably are wider than the central absorbent layer 284 that forms the absorbent core, and their side portions preferably are sealed to one another by bonding, by crimping or by both to prevent release of opened tow and particles of SAP. In addition, the upper and lower layers 280, 282, preferably are comprised of the same material folded over onto itself, and only the open end sealed by crimping or bonding. The absorbent core 28, comprising the assembly of the upper and lower layers 280, 282 and the central absorbent layer 284 including the opened tow and SAP, may be further processed as it is conveyed through the assembly line for inclusion into absorbent garments. For example, the absorbent core 28 may be severed into individual absorbent cores, and the severed ends may be crimped or bonded or both to prevent the SAP from exiting the ends.

Crimping, bonding or both can be performed on the absorbent core 28 of the invention using conventional means. For example, the lateral side edges, and longitudinal edges can be sealed together by intermittent or continuous application of adhesive to the respective portions of the upper and lower layers 280, 282 using any device capable of applying adhesives to a continuous moving web of material. The lateral and/or longitudinal edges then can be pressed together to form a seal. The seal also can be formed ultrasonically, or the respective edges (lateral and/or longitudinal) can be crimped using crimping rollers or any other crimping device known to those having ordinary skill in the art. Using the guidelines provided herein, those skilled in the art will be capable of sealing the lateral and/or longitudinal edges of absorbent core 28 using bonding, crimping, or both.

As shown in FIG. 5, the tow fibers 288 and SAP material 286 are present in a greater amount in the microwells 310, optionally where adhesive has been applied. While not intending on being bound by any theory, the present inventors believe that the median pore size of the porous matrix of tow fibers is greater than the mean diameter of the SAP particles 286 thereby allowing the SAP particles 286 to migrate until they are affixed in position by compression, or other means known in the art. Thus, the SAP particles 286 are free to move about in absorbent core 28 for a given period of time during manufacture. When the SAP particles 286 encounter the microwells 310, (and optional adhesive) they will tend to stop migration and remain in place. The same holds true for the individual fibers 288 that make up the porous fibrous matrix of tow fibers. They too will move about for the same given period of time during manufacture, and will tend to stop migration and remain in place at or about the location of the adhesive 295.

The microwells 310 can be formed of any cross-sectional shape and size. It is preferred that the microwells 310 have a generally circular cross-sectional shape (e.g., circular, oval, polygonal, etc.). It is preferred that the width of the major dimension of the microwell cross-section be within the range of from about 0.5 mm to about 10 mm, more preferably from about 1 mm to about 7 mm, and most preferably from about 2 mm to about 4 mm. It also is preferred that the depth of the average microwell be within the range of from about 0.25 mm to about 5 mm, more preferably from about 0.5 mm to about 4 mm, and most preferably from about 1 mm to about 3 mm.

It is possible in the present invention that the absorbent core 28 be folded as it is disposed between the top sheet 24 and back sheet 26. The absorbent core 28 can be folded in any suitable manner, including any and all of those disclosed in U.S. Pat. No. 6,068,620. Suitable folds include “C” folds, “G” folds, “U” folds, “A” folds, pleats or “W” folds, and the like.

The invention also relates to a method of making an absorbent core, and an absorbent article that includes providing a top sheet material 24 and a back sheet material 26. The method also includes preparing an absorbent core 28 by disposing a central fibrous layer comprising a mixture of tow fibers and SAP between an upper layer 280 and a lower layer 282, whereby at least one of the upper layer 280 and/or lower layer 282 includes a plurality of microwells. The method includes disposing the absorbent core 28 between the top sheet 24 and the back sheet 26. The method provides an absorbent core 28 having select regions of increased absorbency due to the presence of varying concentrations of SAP. Alternatively, the microwells can be formed in a uniform fashion to provide relatively consistent absorption throughout the cross-section of the core 28.

FIG. 2 illustrates an apparatus useful in forming an absorbent article 10 in accordance with the present invention. Any type of tow fiber 288, or mixtures of tow fibers 288, can be supplied to the apparatus and, as conventional in the art, the tow fiber 288 typically is opened prior to forming a fibrous matrix. In this regard, the apparatus includes a tow opener and feeder 810 that is capable of opening any suitable tow material, expanding the tow fiber and feeding the tow fiber to the core forming station 820. Any suitable tow opener and feeder 810 can be used in the method of the invention. Preferably, the tow opener and feeder 810 is capable of opening a plurality of different tow fibers (e.g., varying denier, coarseness, chemical make-up, etc.) and feeding the fibers to the core forming station 820. For example, the apparatus may include two or more tow opener devices 810, that feed the tow to a common nozzle (not shown) that distributes the combined tow fibers to the core forming station 820.

The tow fibers 288 preferably are mixed with superabsorbent polymer (SAP) material 286 to form central fibrous layer 284. The SAP is fed to the core forming station 820 by any SAP feeder 860 capable of feeding the SAP to the core forming station 820. Those skilled in the art are capable of designing a suitable SAP feeder 860 and nozzle configuration to provide adequate mixing of SAP material 286 and tow fibers 288 to form central fibrous layer 284.

Absorbent core 28 can be formed at core forming station 820, where central fibrous layer 284, comprised of SAP material 286 and tow fibers 288, is disposed between an upper layer 280, and a lower layer 282. Upper and lower layers 280, 282 can be fed to core forming unit 820 using any supplying mechanism known in the art, and preferably are fed through one or more feed rollers. The apparatus in FIG. 2 illustrates an alternative embodiment where upper and lower layers 280, 282 are formed from the same material. In this regard, lower layer 282 is fed to core forming station 820 and then folded over onto itself at folding/sealing device 835. Any device capable of folding lower layer 282 onto itself and then optionally sealing the core can be used in the invention. The upper and lower layers 280, 282 can be sealed to one another by use of adhesives from adhesive applicator 840, or by crimping, as is well known in the art.

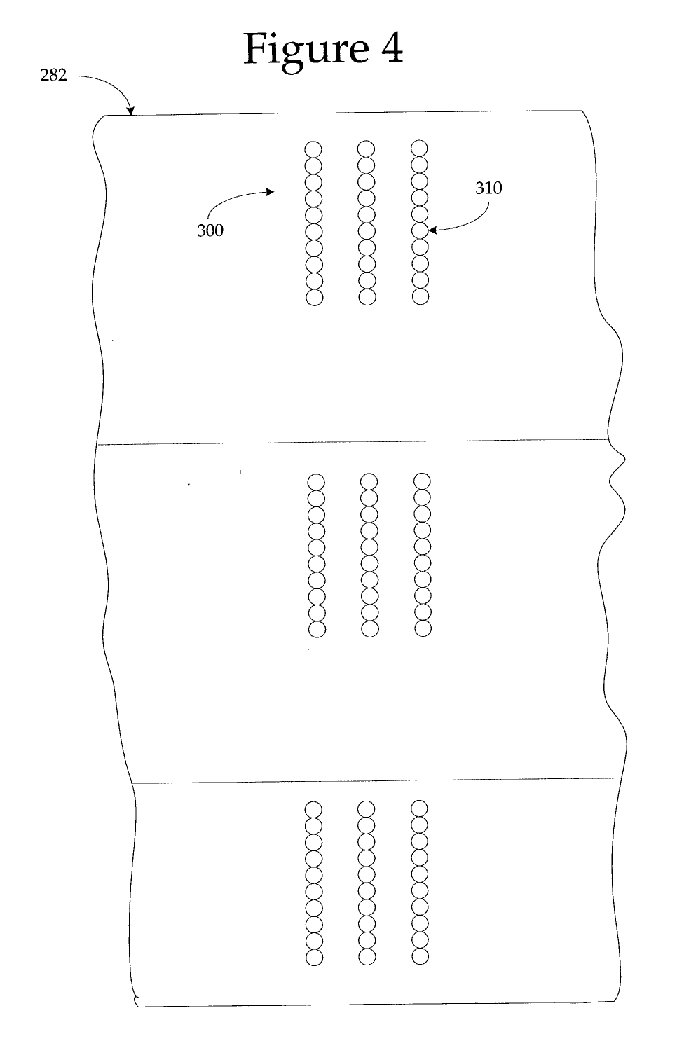

The lower layer 282, or upper layer 280 (not shown in FIG. 2) or both preferably contains a plurality of microwells 310. The microwells can be included in the outer layer prior to feeding the layer to core forming station 820 via feed roller 2820, or they can be formed by, for example, embossing during manufacture of the core. As shown in FIG. 2, an embossing roll 825 can be disposed prior to core forming station 820 to form a plurality of microwells 310. Any pattern can be embossed in outer layer 282 using techniques described, for example, in U.S. Pat. No. 6,461,720, the disclosure of which is incorporated by reference herein in its entirety. Examples of microwell patterns 300 are shown in FIGS. 3 and 4. As shown, the microwell patterns 300 can comprise a series of concentric circles of microwells 310 (FIG. 3), or a series of longitudinal lines or troughs of microwells 310 (FIG. 4). The circles or troughs preferably is present at or near the insult point. Those skilled in the art are capable of designing a suitable embossing roll 825 to form a plurality of microwells 310 in any suitable microwell pattern 300, using the guidelines provided herein.

After the outer layer 282 has been embossed with the plurality of microwells 310, adhesive can be applied by an adhesive applicator 840. Again, any mechanism capable of supplying an adhesive, albeit a spray adhesive, or one that is “rubbed” on, can be used in the invention. Suitable adhesives include any adhesive commonly employed in absorbent garments that is useful in adhering one or more tissue and/or non-woven materials together. It is particularly preferred to use construction adhesives, including HL-1258 by H. B. Fuller Company of St. Paul, Minn.; Findley 2031 and H2587-01 by Ato Findley Inc. of Wauwatosa, Wis.; and NS34-5665 by National Starch Co. of Bridgewater, N.J. Other adhesives that may be used in the invention include 34-578A, available from National Starch Co. of Bridgewater, N.J. The adhesives used in the invention may be used in all adhesive applications in the absorbent garment, or only in select applications as a construction adhesive for bonding parts of the garment as the top sheet, back sheet, absorbent core, and additional layer(s).

The positioning and amount of adhesive can be altered either during line down time, or during manufacture of absorbent article 10, by controlling the positioning of adhesive applicator 840 with an adhesive applicator controller (not shown). Any system can be used to control the amount, if any, and location of application of the adhesive. Those skilled in the art are capable of designing a suitable adhesive applicator 840 to apply select amounts of adhesive, in select positions on either upper and/or lower layers 280, 282, using the guidelines provided herein.

As the SAP material 286 and tow fibers 288 mix together to form central fibrous layer 284, which in turn is disposed on outer layer 282 at core forming station 820, the SAP will preferentially migrate to those regions where the microwells 310 and/or adhesive exists. Some of these SAP particles may become affixed in the microwells 310 and/or adhesive when the absorbent core 28 is passed through the one or more nip rollers 821 at the core forming station 820. The outer layer 282 then can be folded over onto itself and sealed in folding/sealing device 835. The absorbent cores 28 then are cut to length by cutting knife 830. Cutting knife 830 can be any suitable cutting device capable of cutting absorbent core 28 of the invention. For example, cutting knife 830 can be comprised of a set of rollers; one being an anvil, and another having a knife attached at one point on the roller, whereby the diameter of the roller is selected to coordinate with the speed at which absorbent cores 28 are formed. The knife roller and anvil roller then can rotate at the same speed as the line speed to cut the absorbent core 28 at select areas to form uniform length cores 28. Skilled artisans are capable of designing a suitable cutting knife 830 given the specifics of each article forming assembly line.

The absorbent cores 28 then are transported to forming station 800 via core conveyor 880. Top sheet material 24 may be supplied to forming station 800 by top sheet supply mechanism 240, which can be any supply mechanism capable of supplying top sheet 24 to forming station 800. Preferably, top sheet material 24 is supplied via a supply roller 240 and select feed and/or guide rollers (not shown). Back sheet material 26 likewise can be supplied to forming station 800 by back sheet supply mechanism 260, which can be any supply mechanism capable of supplying back sheet 26 to forming station 800. Preferably, back sheet material 26 is supplied via a supply roller 260 and select feed and/or guide rollers (not shown). Forming station brings together the respective components of absorbent article 10 by disposing absorbent core 28 between top sheet material 24, and back sheet material 26. The final absorbent article 10 then may be cut and folded to the appropriate size and shape downstream from forming station 800.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. An absorbent article having a longitudinal dimension and a lateral dimension comprising:
a top sheet;
a back sheet; and
a absorbent core disposed at least partially between the top sheet and the back sheet;
wherein the absorbent core is comprised of:
an upper layer;
a lower layer; and
a central absorbent layer including a mixture of fibrous material and SAP disposed between the upper and lower layer, wherein at least one of the upper layer or the lower layer comprises a plurality of microwells.

2. The absorbent article of claim 1, whereby the article has a first waist region, a second waist region longitudinally opposed to the first waist region, and a crotch region between the first and second waist regions, the article further comprising at least one fastening element attached to a lateral edge of the first waist region; and
one or more target devices attached to the article in the second waist region, where at least one fastening element and the one or more target devices are capable of attaching to one another, the one or more target devices being located so that the first waist region and second waist region of the garment may be joined to one another to secure the garment on a wearer.

3. The absorbent article of claim 2, further comprising elastic leg gathers comprising one or more elastic materials disposed adjacent a lateral edge of the crotch region, and standing leg gathers disposed on the top sheet adjacent the lateral edge of the crotch region.

4. The absorbent article of claim 2, wherein the at least one fastening element comprises a hook portion of a hook and loop fastener and the one or more target devices comprise the loop portion of a hook and loop fastener.

5. The absorbent article of claim 2, wherein the at least one fastening element is an adhesive tape and the one or more target devices comprise a tape receiving surface.

6. The absorbent article of claim 2, wherein the at least one fastening element is comprised of a pair of laterally extending tabs disposed on the lateral edges of the first waist region, whereby the laterally extending tabs each include at least one fastening element.

7. The absorbent article of claim 1, wherein the absorbent core is a multi-layered absorbent core unit including two outer tissue layers and a central fibrous layer that comprises from about 50% to about 95% by weight super absorbent polymer (SAP).

8. The absorbent article of claim 7, wherein the central fibrous layer comprises tow fibers selected from the group consisting of cellulose acetate fibers, polypropylene fibers, polyester fibers, rayon fibers, LYOCELL fibers, polyacrylonitrile fibers, cotton fibers, cotton linter fibers, and mixtures thereof.

9. The absorbent article of claim 7, wherein the central fibrous layer further comprises up to 10% by weight fluff wood pulp fibers.

10. The absorbent article of claim 7, wherein the central fibrous layer further comprises particulate additives.

11. The absorbent article of claim 8, wherein the tow is a cellulose ester tow or a polypropylene tow.

12. The absorbent article of claim 11, wherein the tow is a cellulose acetate tow.

13. The absorbent article of claim 1, wherein the upper and lower layer are comprised of the same material, and the material is selected from the group consisting of tissue, airlaid fluff pulp, synthetic non-woven material, and mixtures or combinations thereof.

14. The absorbent article of claim 13, wherein the upper layer is fluid pervious, and the lower layer is fluid impervious.

15. The absorbent article of claim 1, wherein lateral edges of the absorbent core are crimped.

16. The absorbent article of claim 1, wherein the longitudinal edges of the absorbent core are crimped.

17. The absorbent article of claim 1, wherein the concentration of SAP is greater in the microwell-containing regions of the core, when compared to non-microwell-containing regions of the core.

18. The absorbent article of claim 1, wherein the plurality of microwells are present primarily at or near the insult point of the absorbent core.

19. The absorbent article of claim 1, wherein the at least one of the plurality of microwells comprises an adhesive.

20. The absorbent article of claim 1, wherein the average diameter of the plurality of microwells is within the range of from about to about.

21. The absorbent article of claim 20, wherein the average diameter is within the range of from about to about.

22. The absorbent article of claim 1, wherein the average depth of the microwells is within the range of from about to about.

23. The absorbent article of claim 22, wherein the average depth of the microwells is within the range of from about to about.

24. The absorbent article of claim 8, wherein the tow is chemically modified tow.

25. An absorbent laminate comprising:
an upper layer;
a lower layer; and
a central absorbent layer including a mixture of fibrous material and SAP disposed between the upper and lower layer, wherein at least one of the upper layer or the lower layer comprises a plurality of microwells.

26. The absorbent laminate of claim 25, comprising two outer tissue layers and a central fibrous layer that comprises from about 50% to about 95% by weight super absorbent polymer (SAP).

27. The absorbent laminate of claim 25, wherein the central absorbent layer comprises tow fibers selected from the group consisting of cellulose acetate fibers, polypropylene fibers, polyester fibers, rayon fibers, LYOCELL fibers, polyacrylonitrile fibers, cotton fibers, cotton linter fibers, and mixtures thereof.

28. The absorbent laminate of claim 25, wherein the central absorbent layer further comprises up to 10% by weight fluff wood pulp fibers.

29. The absorbent laminate of claim 25, wherein the central absorbent layer further comprises particulate additives.

30. The absorbent laminate of claim 27, wherein the tow is a cellulose ester tow or a polypropylene tow.

31. The absorbent laminate claim 27, wherein the tow is a cellulose acetate tow.

32. The absorbent laminate of claim 256, wherein the upper and lower layer are comprised of the same material, and the material is selected from the group consisting of tissue, airlaid fluff pulp, synthetic non-woven material, and mixtures or combinations thereof.

33. The absorbent laminate of claim 25, wherein the upper layer is fluid pervious, and the lower layer is fluid impervious.

34. The absorbent laminate of claim 25, wherein adhesives are applied only in its lateral edges.

35. The absorbent laminate of claim 25, wherein the lateral edges of the absorbent laminate are crimped.

36. The absorbent laminate of claim 25, wherein longitudinal edges of the absorbent laminate are crimped.

37. The absorbent laminate of claim 25, wherein the concentration of SAP is greater in the microwell-containing regions of the core, when compared to non-microwell-containing regions of the core.

38. The absorbent laminate of claim 25, wherein the plurality of microwells are present primarily at or near the insult point of the absorbent core.

39. The absorbent laminate of claim 25, wherein the at least one of the plurality of microwells comprises an adhesive.

40. The absorbent laminate of claim 25, wherein the average diameter of the plurality of microwells is within the range of from about to about.

41. The absorbent laminate of claim 40, wherein the average diameter is within the range of from about to about.

42. The absorbent laminate of claim 25, wherein the average depth of the microwells is within the range of from about to about.

43. The absorbent laminate of claim 42, wherein the average depth of the microwells is within the range of from about to about.

44. A method of making an absorbent article comprising:
a) preparing a top sheet and a back sheet;
b) preparing an absorbent core by:
b1) providing an upper layer;
b2) providing a lower layer;
b3) providing a central absorbent layer that comprises a mixture of fibrous material and superabsorbent polymer (SAP); and
b4) disposing the central absorbent layer at least partially between the upper layer and the lower layer; and
c) disposing the absorbent core at least partially between the top sheet and the back sheet,
wherein at least one of the upper layer or the lower layer comprises a plurality of microwells.

45. The method of claim 44, wherein preparing the absorbent core further comprises supplying select regions of adhesive to at least one of the upper and lower layers prior to disposing the central absorbent layer at least partially between the upper layer and the lower layer.

46. The method of claim 44, wherein preparing the absorbent core comprises supplying no adhesive to the upper or lower layer thereby providing a gravitationally zoned absorbent core.

47. The method of claim 44, further comprising embossing at least the upper layer or the lower layer with a plurality of microwells prior to disposing the central absorbent layer between the upper layer and the lower layer.

48. The method of claim 45, wherein the select regions of adhesives correspond to the location of at least one of the plurality of microwells.

49. The method of claim 47, wherein the plurality of microwells are formed in such a manner that the plurality of microwells are present primarily at or near the insult point of the absorbent article.

50. The method of claim 44, further comprising crimping lateral edges of the absorbent core.

51. The method of claim 44, further comprising crimping longitudinal edges of the absorbent core.

52. A method of making an absorbent laminate comprising:
providing an upper layer;
providing a lower layer;
providing a central absorbent layer that comprises a mixture of tow fibers and superabsorbent polymer (SAP); and
disposing the central absorbent layer at least partially between the upper layer and the lower layer,
wherein at least one of the upper layer or the lower layer comprises a plurality of microwells.

53. The method of claim 52, wherein preparing the absorbent core further comprises supplying select regions of adhesive to at least one of the upper and lower layers prior to disposing the central absorbent layer at least partially between the upper layer and the lower layer.

54. The method of claim 52, wherein preparing the absorbent core comprises supplying no adhesive to the upper or lower layer thereby providing a gravitationally zoned absorbent core.

55. The method of claim 52, further comprising embossing at least the upper layer or the lower layer with a plurality of microwells prior to disposing the central absorbent layer between the upper layer and the lower layer.

56. The method of claim 53, wherein the select regions of adhesives correspond to the location of at least one of the plurality of microwells.

57. The method of claim 55, wherein the plurality of microwells are formed in such a manner that the plurality of microwells are present primarily at or near the insult point of the absorbent article.

58. The method of claim 52, further comprising crimping lateral edges of the absorbent core.

59. The method of claim 52, further comprising crimping longitudinal edges of the absorbent core.
